(19) United States
(12) Patent Application Publication
(10) Pub. No.: US 2022/0333094 A1
(43) Pub. Date: Oct. 20, 2022

(54) METHOD FOR PREPARING CELL MEMBRANE-COATED NANO TOPOLOGICALARRAY AND USE THEREOF

(71) Applicant: SOOCHOW UNIVERSITY, Suzhou (CN)

(72) Inventors: Jian LIU, Suzhou (CN); Sidi LIU, Suzhou (CN); Xiang XIAO, Suzhou (CN)

(21) Appl. No.: 17/762,349

(22) PCT Filed: Feb. 4, 2021
(86) PCT No.: PCT/CN2021/075161

§ 371 Date: Mar. 21, 2022

(30) Foreign Application Priority Data
Jun. 24, 2020 (CN) 202010590283.5

(51) Int. Cl. C12N 11/14; C12N 5/0786; C12N 5/00; A61L 15/40; A61K 35/15

(52) U.S. Cl. CPC C12N 11/14; C12N 5/0645; C12N 5/0006; A61L 15/40; A61K 35/15; C12N 2502/70; A61L 2400/12; A61L 2420/02

(57) ABSTRACT

A method for preparing a cell membrane-coated nano topological array and a use thereof are disclosed. The method includes: stimulating macrophages to form stimulated macrophages, and extracting the membrane of the stimulated macrophages; at the same time, processing a substrate to form a substrate with nanowires, and treating the substrate with nanowires to form a positively charged nanowire substrate; combining the membrane of the stimulated macrophages with the positively charged nanowire substrate to obtain a macrophage membrane-modified nano topological array. The present invention is simple in preparation and operation, and can be applied to capture bacteria.

### FIELD OF THE INVENTION

The present invention relates to the technical field of cell membrane-encapsulated nanoparticles, and more particularly to a method for preparing a cell membrane-coated nano topological array, and a use thereof.

### DESCRIPTION OF THE RELATED ART

Chemically modified functional biological proteins on the topological structure surface of a material is used to prepare a platform for capturing of bacteria and efficient monitoring. For example, Xiaodong Chen et al. (Yong-Qiang Li, Bowen Zhu, Yuanang Li, Wan Ru Leow, Rubyn Goh, Bing Ma, Eileen Fong, Mark Tang, and Xiaodong Chen; A Synergistic Capture Strategy for Enhanced Detection and Elimination of Bacteria. Angewandte Chemie 2014, 5947) modified the surface of a silicon nanowire array with lysozyme or concanavalin A to achieve efficient capturing and killing of bacteria.

Although the above method can efficiently capture and kill bacteria, it uses chemical bonding to modify the protein on the surface of the material, which requires a complex modification process and cannot guarantee that the modified protein has ideal spatial shape; in addition, there are shortcomings such as difficulty in material preparation, complicated and time-consuming operation, and limited application.

### SUMMARY OF THE INVENTION

In view of the above, the technical problem to be solved by the present invention is to overcome the difficulty in material preparation, complicated and time-consuming operation, and limited application in the prior art, thereby providing a method for preparing a cell membrane-coated nano topological array, which has simple preparation and operation and limited application, and a use thereof.

In order to solve the above technical problem, a method for preparing a cell membrane-coated nano topological array, includes the following steps: stimulating macrophages to form stimulated macrophages, and extracting the membrane of the stimulated macrophages; and processing a substrate to form a substrate with nanowires, and treating the substrate with nanowires to form a positively charged nanowire substrate; combining the membrane of the stimulated macrophages with the positively charged nanowire substrate to obtain a macrophage membrane-modified nano topological array, where after the nano topological array is coated, functional proteins on the membrane surface retain the original biological spatial structure, biological activity and biological function.

In an embodiment of the present invention, extracting the membrane of the stimulated macrophages includes: repeatedly freeze thawing the stimulated macrophages, and performing high-speed centrifugation on the thawed liquid in a centrifuge, collecting a precipitate and adding a new buffer solution is added thereto, to obtain a cell membrane suspension.

In an embodiment of the present invention, when the stimulated macrophages are thawed, after the liquid is thawed well, it is transferred into liquid nitrogen again, and after repeated operations, the thawed liquid is put into the centrifuge.

In an embodiment of the present invention, treating the substrate with nanowires to form a positively charged nanowire substrate includes arranging a silane coupling agent on the substrate with nanowires.

In an embodiment of the present invention, before arranging a silane coupling agent on the substrate with nanowires, the method also includes sequentially immersing the substrate in an aqueous solution of hydrofluoric acid, a solution of silver nitrate and hydrofluoric acid, and a nitric acid solution, and then washing the substrate with deionized water and blow drying the substrate with nitrogen.

In an embodiment of the present invention, arranging a silane coupling agent on the substrate with nanowires includes: treating the substrate with nanowires with an oxygen plasma cleaner at a high power for a set time, and immersing the treated substrate in an absolute ethanol solution containing a specified concentration of APTES within a specified time.

The present invention also provides use of a cell membrane-coated nano topological array. A macrophage membrane-modified silicon nano topological array is obtained by the method for preparing a cell membrane-coated nano topological array, and the macrophage membrane-modified nano topological array is used as a base for a medical dressing to cover a culture plate containing colonies or a wound infection site.

The present invention also provides a use of a cell membrane-coated nano topological array, including: manufacturing a microfluidic chip, with a chip channel disposed in the microfluidic chip; and combining the microfluidic chip with a macrophage membrane-modified silicon nano topological array obtained by the method for preparing a cell membrane-coated nano topological array; and injecting a liquid sample into the chip channel to specifically capture and eliminate bacteria.

In an embodiment of the present invention, manufacturing a microfluidic chip comprises preparing a PDMS prepolymerization solution and pouring the PDMS prepolymerization solution on a carrier containing a microfluidic chip pattern, curing the carrier, and peeling off the carrier after forming to obtain a microfluidic chip.

In an embodiment of the present invention, a microfluidic catheter is arranged at the inlet and outlet of the chip channel, and the liquid sample is injected into the chip channel through the microfluidic catheter.

As compared with the prior art, the above-mentioned technical solutions of the present invention has the following advantages:

According to the preparation method and use of the cell membrane-coated nano topological array of the present invention, the protein expression on the cell membrane of stimulated cells is changed through external stimulus, thereby obtaining a cell membrane enriched with target proteins; the cell membrane is coated on the surface of a material with a nano topological structure through electrostatic interactions, where the coated cell membrane has cell membrane fluidity, and the protein expressed on the cell membrane has the original biological spatial structure, biological activity and biological function; the nano topological array coated with the membrane of macrophages stimulated by bacteria can efficiently capture the attached bacteria under static conditions, so the preparation and operation are simple; in addition, a chip assembled from a base of a material of a cell membrane-modified nano topological structure and a patterned microfluidic channel can be applied in the capture of bacteria.

### BRIEF DESCRIPTION OF THE DRAWINGS

In order to make the present invention easy to be understood clearly, the present invention is further explained in detail below according to specific embodiments of the present invention and in conjunction with the accompanying drawings.

FIG. 1 is a flow chart of a method for preparing a cell membrane-coated nano topological array of the present invention;

FIG. 2a is a transmission electron micrograph of a nanowire without cell membrane modification according to the present invention;

FIG. 2b is a transmission electron micrograph of a nanowire with cell membrane modification according to the present invention;

FIG. 3 is a schematic diagram of a positively charged nanowire substrate according to the present invention;

FIG. 4 is a three-dimensional view of a microfluidic device combined with macrophage membrane modification according to the present invention;

FIG. 5 is the statistics of the capture of bacteria in blood mixed with different amounts of Staphylococcus aureus by a microfluidic chip according to the present invention;

FIG. 6 is the statistics of the capture of bacteria in blood mixed with different amounts of Escherichia coli by a microfluidic chip according to the present invention;

Reference numerals: 10-macrophage, 11-stimulated macrophage, 12-membrane of stimulated macrophages, 20-substrate, 21-substrate with nanowires, 22-positively charged nanowire substrate, 30-macrophage membrane-modified nano topological array, 40-microfluidic device, 41-microfluidic catheter.

### DETAILED DESCRIPTION OF THE PREFERRED EMBODIMENTS

### Embodiment 1

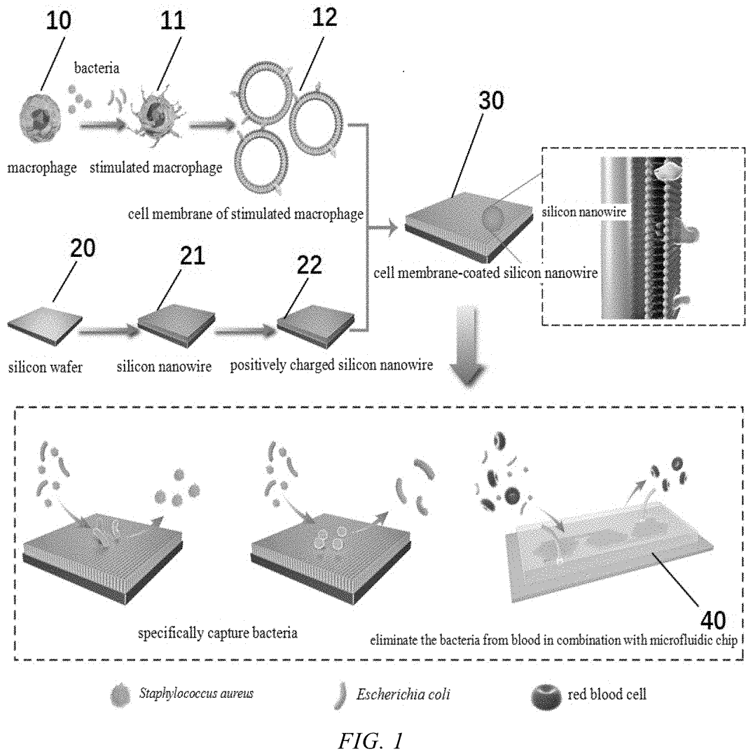

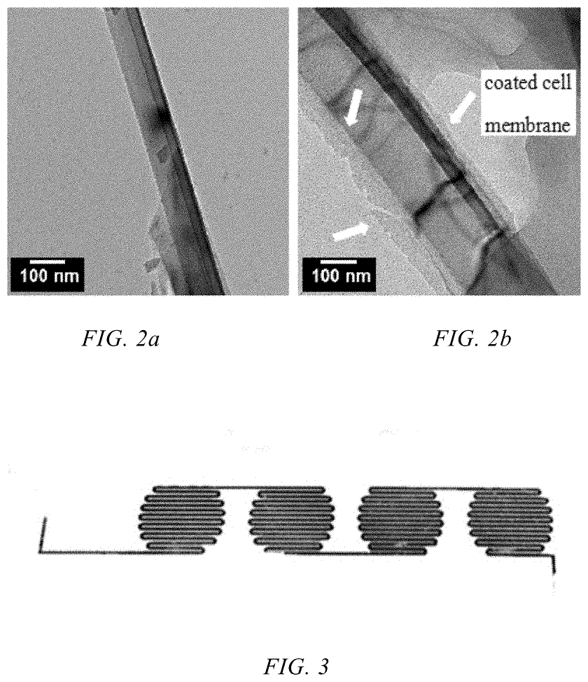

As shown in FIG. 1, this embodiment provides a method for preparing a cell membrane-coated nano topological array, including the following steps: step S1: macrophages 10 were stimulated to form stimulated macrophages 11, and the membrane 12 of the stimulated macrophages was extracted; at the same time, a substrate 20 was processed to form a substrate with nanowires 21, as shown FIG. 2a, and the substrate was treated with nanowires 21 to form a positively charged nanowire substrate 22, as shown FIG. 3; step S2: the membrane 12 of the stimulated macrophages was combined with the positively charged nanowire substrate 22, as shown FIG. 2b, to obtain a macrophage membrane-modified nano topological array 30.

In the method for preparing a cell membrane-coated nano topological array of this embodiment, in the step S1, macrophages 10 were stimulated to form stimulated macrophages 11, and the membrane 12 of the stimulated macrophages was extracted, so that the macrophage membrane was negatively charged; at the same time, a substrate 20 was processed to form a substrate with nanowires 21, as shown in FIG. 2a, and the substrate with nanowires 21 was treated to form a positively charged nanowire substrate 22, as shown in FIG. 3; in the step S2, the membrane 12 of the stimulated macrophages was combined with the positively charged nanowire substrate 22, as shown in FIG. 2b, to obtain a macrophage membrane-modified silicon nano topological array 30. After the nano topological array 30 is coated, functional proteins on the membrane surface retain the original biological space structure, biological activity and biological function. Since the cell membrane is coated on the surface of the structured substrate by means of the charge effect, the coated cell membrane has cell membrane fluidity, and biologically functional proteins on the cell membrane have the original spatial morphology. In addition, the macrophage membrane-modified nano topological array 30 is a base with strong bacteria capture ability, which facilitates efficient elimination of bacteria from blood contaminated by bacteria.

A method for extracting the membrane 12 of the stimulated macrophages is as follows: the stimulated macrophages 11 are repeatedly frozen and thawed, the thawed liquid is subjected to high-speed centrifugation in a centrifuge, a precipitate is collected and a new buffer solution is added thereto, that is, a cell membrane suspension is obtained, thereby extracting the membrane of the stimulated macrophages.

When the stimulated macrophages 11 are thawed, after the liquid is thawed well, it is transferred into liquid nitrogen again, liquid nitrogen will freeze the cells instantaneously and the cells will be fragmented by thawing again, and after repeated operations, the thawed liquid, namely, the suspended cell membrane, is put into the centrifuge where it is subjected to high-speed centrifugation.

The following details how to extract the membrane 12 of the stimulated macrophages:

Mouse-derived macrophages J774A.1 were cultured in a 100 mm culture dish, and once the number of cell proliferation reached 1 million, 5 million bacteria were added into the plate to stimulate for 3 h.

After the stimulation by bacteria was completed, the cells were washed with a phosphate buffer solution. After washing, macrophages 10 were rinsed off with the buffer solution and collected, to obtain stimulated macrophages 11.

The stimulated macrophages 11 were transferred into a 1 mL eppendorf (EP) tube and sealed, and the Ep tube was immersed into liquid nitrogen for 10 sec, and then quickly transferred to warm water at 37° C., until the liquid was thawed well, and the Ep tube was transferred to liquid nitrogen again. This operation was repeated 10 times. Because liquid nitrogen would freeze the cells instantaneously, and the cells would be fragmented by thawing again, a thawed liquid could be formed by repeating this process.

The thawed liquid was put into a centrifuge for high-speed centrifugation at a speed of 20,000 gravitational acceleration, a precipitate was collected and a new buffer solution was added thereto, and this operation was repeated 5 times.

The collected precipitate, namely, a cell membrane suspension, was stored in a refrigerator at 4° C. to obtain membranes 12 of stimulated macrophages.

A method for treating the substrate with nanowires 21 to form a positively charged nanowire substrate 22 is as follows: a silane coupling agent is arranged on the substrate with nanowires 21, so that positive charges are formed on the substrate with nanowires 21, which facilitates adsorption of the membrane 12 of the stimulated macrophages by means of the electrostatic effect.

Before arranging a silane coupling agent on the substrate with nanowires 21, the substrate 20 is sequentially immersed in an aqueous solution of hydrofluoric acid, a solution of silver nitrate and hydrofluoric acid, and a nitric acid solution, and then the substrate 20 is washed with deionized water and blown dry with nitrogen. When the substrate 20 is immersed in an aqueous solution of hydrofluoric acid, since the surface of the substrate is usually covered with a layer of silicon dioxide, which can affect the reaction of the substrate 20, this layer of silicon dioxide is corroded by the hydrofluoric acid, so that the substrate 20 itself is exposed. When the substrate 20 is immersed in a solution of silver nitrate and hydrofluoric acid, in the presence of silver nitrate, hydrofluoric acid in combination with silver nitrate can corrode the substrate 20, so that nanowires are generated on the surface. When the substrate 20 is immersed in a nitric acid solution, this facilitates removal of residual chemical substances remaining on the surface of the substrate 20.

A method for arranging a silane coupling agent on the substrate with nanowires 21 is as follows: the substrate with nanowires 21 is treated with an oxygen plasma cleaner at a high power for a set time, and immersed in an absolute ethanol solution containing a specified concentration of APTES within a specified time.

The method for processing a substrate 20 to form a substrate with nanowires 21 is as follows: a layer of positive photoresist was spin-coated on the surface of the substrate 20; and the substrate 20 coated with the photoresist is irradiated with ultraviolet using a mask; the irradiated substrate 20 is quickly rinsed with a developer solution to obtain a photoresist pattern to form a substrate with nanowires 21.

The following details how to form the positively charged nanowire substrate 22.

A layer of positive photoresist was spin-coated on a smooth surface of a substrate 20 which was washed with a piranha solution.

the substrate 20 coated with the photoresist was irradiated with ultraviolet using a mask;

the irradiated substrate was quickly rinsed with a developer solution to obtain a photoresist pattern to form a substrate with nanowires 21, thereby obtaining a texture to facilitate cooperation with a microfluidic chip;

this layer of patterned photoresist was used as a mask and immersed in an aqueous solution containing 5% hydrofluoric acid for 5 min, where the mask can block some areas, unblocked areas are exposed out of the substrate, and the exposed part is the pattern on the chip; the substrate 20 was removed and immersed in a solution containing 0.02M silver nitrate and 4.8M hydrofluoric acid for 40 min; therefore, the exposed substrate was etched by silver nitrate and hydrofluoric acid to obtain the substrate with nanowires 21;

the substrate with nanowires 21 was transferred to a 50% nitric acid solution and immersed therein for 20 min, to facilitate removal of residual chemical substances remaining on the surface of the substrate with nanowires 21;

the substrate with nanowires 21 was washed with deionized water and blown dry with nitrogen; specifically, the substrate with nanowires 21 was immersed in an acetone solution to remove the photoresist coating and washed with deionized water, and finally blown dry with nitrogen, where only when nanowires are obtained on the texture, they will come into contact with the flowing part of the chip, which facilitates modification of the cell membrane.

The dried substrate with nanowires 21 was treated with an oxygen plasma cleaner at high power for 3 min and immersed in an absolute ethanol solution containing 10% APTES for 10 min, thereby forming a positively charged nanowire substrate 22.

In this embodiment, the substrate 20 can be a silicon wafer.

### Embodiment 2

As shown in FIG. 1, this embodiment provides a first example of using a cell membrane-coated nano topological array. A macrophage membrane-modified nano topological array 30 is obtained by the method for preparing a cell membrane-coated nano topological array described in embodiment 1, and the macrophage membrane-modified nano topological array 30 is used as a base for a medical dressing to cover a culture plate containing colonies.

This embodiment provides a first example of using a cell membrane-coated nano topological array. A macrophage membrane-modified nano topological array 30 is obtained by the method for preparing a cell membrane-coated nano topological array described in embodiment 1, and the macrophage membrane-modified nano topological array 30 is used as a base for a medical dressing to cover a culture plate containing colonies or a wound infection site, which facilitates observation of the captured bacteria.

Specifically, Staphylococcus aureus or Escherichia coli was coated on a culture plate and colonies were formed; the macrophage membrane-modified nano topological array 30 was used as a base for a medical dressing to cover a culture plate containing colonies; after a period of time, the base was removed and the captured bacteria were observed, to facilitate subsequent analysis and research.

### Embodiment 3

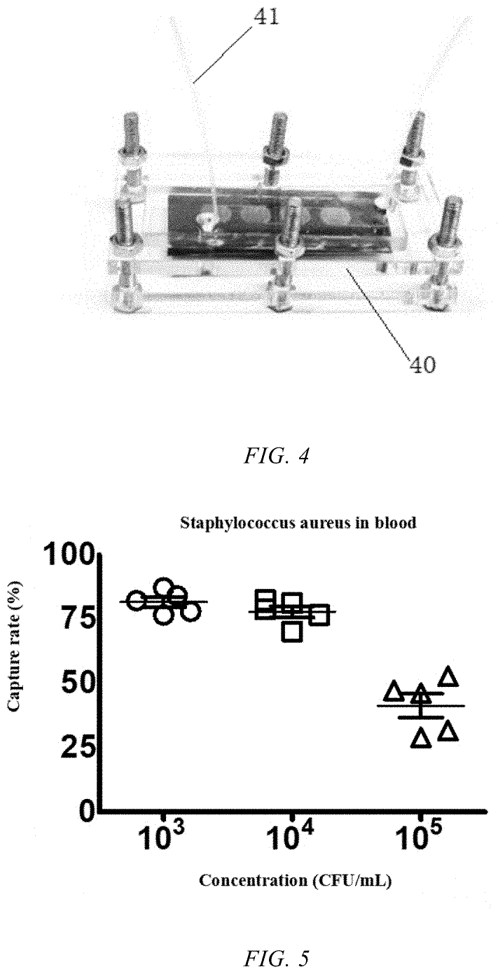

As shown in FIGS. 1 and 3, this embodiment provides a second example of using a cell membrane-coated nano topological array, including: manufacturing a microfluidic chip, with a chip channel disposed in the microfluidic chip; and combining the microfluidic chip with a macrophage membrane-modified nano topological array 30 obtained by the method for preparing a cell membrane-coated nano topological array described in embodiment 1, as shown in FIG. 4; and injecting a liquid sample into the chip channel to specifically capture and eliminate bacteria.

This embodiment provides a second example of using a cell membrane-coated nano topological array, including: manufacturing a microfluidic chip, with a chip channel disposed in the microfluidic chip, to facilitate injection of a liquid sample into the chip channel; combining the microfluidic chip with a macrophage membrane-modified nano topological array 30 obtained by the method for preparing a cell membrane-coated nano topological array described in embodiment 1, thereby forming a microfluidic device with a nano topological array 40; a liquid sample was injected into the chip channel to specifically capture and eliminate bacteria. The microfluidic device 40 can specifically capture and eliminate bacteria while flowing in the chip channel, so that bacteria in blood contaminated by bacteria can be efficiently removed, thereby facilitating subsequent analysis and research.

A method for manufacturing the microfluidic chip is as follows: a PDMS prepolymerization solution is prepared and poured on a carrier containing a microfluidic chip pattern, the carrier is cured, and after forming, the carrier is peeled off to obtain a microfluidic chip.

Specifically, a PDMS prepolymerization solution was prepared by mixing a prepolymer A and a prepolymer B in a weight ratio of 10 to 1 and poured on a carrier containing a microfluidic chip pattern, and the carrier was transferred into a 70° oven and cured for 40 min, and after curing, the carrier was peeled from the formed PDMS to obtain a microfluidic chip.

A microfluidic catheter 41 is arranged at the inlet and outlet of the chip channel, and the liquid sample is injected into the chip channel through the microfluidic catheter 41. Specifically, a 1 mm round hole was punched at the inlet and outlet of the chip channel and the microfluidic catheter 41 was inserted.

In addition, as shown in FIG. 4, in a specific application process, the microfluidic chip and the macrophage membrane-modified nano topological array 30 are combined together and fixed with a splint with screws to form a microfluidic device 40.

Since in the microfluidic device 40, the nano topological array 30 is without cell membrane modification, it is necessary to introduce the cell membrane for modification therein, and then the cell membrane not used for modification is washed away, so that the cell membrane coating for subsequent experiments can be obtained. Specifically, a cell membrane suspension was ultrasonically dispersed, the dispersed suspension was injected into a microfluidic chip, and the microfluidic chip was placed in a refrigerator at 4° C. overnight while keeping the channel filled with liquid, thereby achieving macrophage membrane modification. It can be ensured by the above introduction of the cell membrane that the cell membrane is only modified in the corresponding area of the chip instead of the entire substrate.

After overnight, the channel of the microfluidic chip was washed with a buffer solution, so as to facilitate washing of the microfluidic chip.

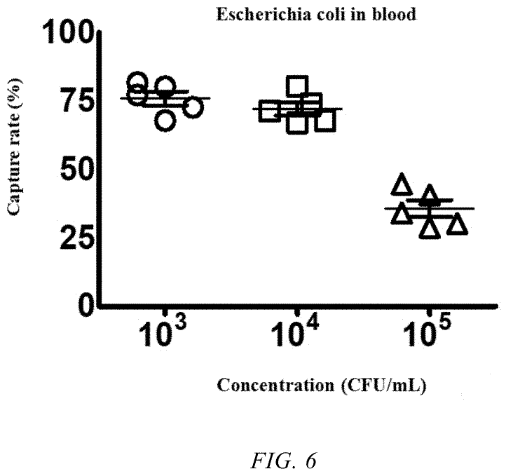

1,000, 10,000, and 100,000 Staphylococcus aureus or Escherichia coli were mixed into 1 mL of mouse blood containing heparin, respectively; the bacteria-contaminated blood was injected into the microfluidic chip at a rate of 0.2 mL per hour; the outflowing blood was collected and the counting experiment was performed to obtain the residual bacterial content in the blood, so as to verify the blood clearance effect, as shown in FIGS. 5 and 6.

Obviously, the foregoing embodiments are merely examples for clear description, and are not intended to limit the implementations. Other changes or variations in different forms can be made for those of ordinary skill in the art on the basis of the above description. It is unnecessary and impossible to list all the implementations here. The obvious changes or variations thus derived are still within the protection scope of the present invention.

1. A method for preparing a cell membrane-coated nano topological array, comprising steps of:
step S1: stimulating macrophages to form stimulated macrophages, and extracting the membrane of the stimulated macrophages; and processing a substrate to form a substrate with nanowires, and treating the substrate with nanowires to form a positively charged nanowire substrate;
step S2: combining the membrane of the stimulated macrophages with the positively charged nanowire substrate to obtain a macrophage membrane-modified nano topological array, wherein after the nano topological array is coated, functional proteins on the membrane surface retain the original biological spatial structure, biological activity and biological function.

2. The method for preparing a cell membrane-coated nano topological array according to claim 1, wherein extracting the membrane of the stimulated macrophages comprises: repeatedly freeze thawing the stimulated macrophages, and performing high speed centrifugation on the thawed liquid; collecting a precipitate and adding a new buffer solution thereto, to obtain a cell membrane suspension.

3. The method for preparing a cell membrane-coated nano topological array according to claim 2, wherein when the stimulated macrophages are thawed, after the liquid is thawed well, it is transferred into liquid nitrogen again, and after repeated operations, the thawed liquid is put into the centrifuge.

4. The method for preparing a cell membrane-coated nano topological array according to claim 1, wherein treating the substrate with nanowires to form a positively charged nanowire substrate comprises arranging a silane coupling agent on the substrate with nanowires.

5. The method for preparing a cell membrane-coated nano topological array according to claim 4, wherein before arranging a silane coupling agent on the substrate with nanowires, the method also comprises sequentially immersing the substrate in an aqueous solution of hydrofluoric acid, a solution of silver nitrate and hydrofluoric acid, and a nitric acid solution, and then washing the substrate with deionized water and blow drying the substrate with nitrogen.

6. The method for preparing a cell membrane-coated nano topological array according to claim 4, wherein arranging a silane coupling agent on the substrate with nanowires comprises: treating the substrate with nanowires with an oxygen plasma cleaner at a high power for a set time, and immersing the treated substrate in an absolute ethanol solution containing a specified concentration of APTES within a specified time.

7. Use of a cell membrane-coated nano topological array, wherein a macrophage membrane-modified nano topological array is obtained by the method for preparing a cell membrane-coated nano topological array according to claim 1, and the macrophage membrane-modified nano topological array is used as a base for a medical dressing to cover a culture plate containing colonies or a wound infection site.

8. A use of a cell membrane-coated nano topological array, comprising manufacturing a microfluidic chip, with a chip channel disposed in the microfluidic chip; and combining the microfluidic chip with a macrophage membrane-modified nano topological array obtained by the method for preparing a cell membrane-coated nano topological array of claim 1; and injecting a liquid sample into the chip channel to specifically capture and eliminate bacteria.

9. The use of a cell membrane-coated nano topological array according to claim 8, wherein manufacturing a microfluidic chip comprises: preparing a PDMS prepolymerization solution and pouring the PDMS prepolymerization solution on a carrier containing a microfluidic chip pattern, curing the carrier, and peeling off the carrier after forming to obtain a microfluidic chip.

10. The use of the cell membrane-coated nano topological array according to claim 8, wherein a microfluidic catheter is arranged at an inlet and outlet of the chip channel, and the liquid sample is injected into the chip channel through the microfluidic catheter.
